(19) United States
(12) Patent Application Publication
(10) Pub. No.: US 2005/0123455 A1
(43) Pub. Date: Jun. 9, 2005

(54) Ozone purifier for vehicle

(75) Inventors: Koichi Inaba, Saitama (JP); Tatsuya Okayama, Saitama (JP); Tsuyoshi Eguchi, Saitama (JP); Atsuhiro Miyauchi, Saitama (JP)

Correspondence Address: ARENT FOX KINTNER PLOTKIN & KAHN, 1050 CONNECTICUT AVENUE, N.W., SUITE 400, WASHINGTON, DC, 20036, US

(73) Assignee: Honda Motor Co., Ltd.

(21) Appl. No.: 10/994,301

(22) Filed: Nov. 23, 2004

(30) Foreign Application Priority Data
Dec. 5, 2003 (JP) 2003-407967

(51) Int. Cl. A61L 9/00

(52) U.S. Cl. 422/120

(57) ABSTRACT

An ozone purifier for a vehicle is configured of an ozone purification catalyst mounted on the vehicle; a first ozone concentration detection mechanism for detecting an ozone concentration in air before its passing the ozone purification catalyst; a second ozone concentration detection mechanism for detecting an ozone concentration in air after its passing the ozone purification catalyst; a deterioration detection mechanism for using output values of the first and second ozone concentration detection mechanisms and detecting deterioration of the ozone purification catalyst; and furthermore a mechanism for calibrating the output values of the first and second ozone concentration detection mechanisms, and when detecting the deterioration of the ozone purification catalyst, the purifier calibrates the output values of the first and second ozone concentration detection mechanisms.

### BACKGROUND OF THE INVENTION

1. Field of the Invention

The present invention relates to an ozone purifier for a vehicle, and particularly, to the ozone purifier for the vehicle for monitoring a performance of the ozone purifier by an onboard diagnosis apparatus (OBD) mounted on the vehicle. Here the onboard diagnosis apparatus (OBD) is a system that is mainly installed in a computer mounted on the vehicle and consists of a software for detecting a malfunction of an emission control system, which generates exhaust of a hazardous substance, by monitoring almost all components and systems and warning a vehicle proprietor by lighting a malfunction indicator light (MIL) on an instrument board when a malfunction with respect to the exhaust is detected.

2. Description of the Related Art

Many countries perform various regulations of a gas (exhaust gas) exhausted from a vehicle. For example, California State in the U.S. performs an exhaust regulation of non-methane organic gases (NMOG), carbon monoxide (CO), nitrogen oxides (NOx), and the like. According to the regulation, each manufacturer selling a vehicle in California State is obligated to observe an exhaust regulation value (regulation value of each vehicle and that of the manufacturer as average) of the NMOG and the like. Therefore, each manufacturer is actively developing an LEV (Low Emission Vehicle) and a ZEV (Zero Emission Vehicle).

Incidentally, such the exhaust regulation gradually tends to be made severe from a consciousness enhancement for an environment in these years. On the other hand, there is also an example of providing a system, considering a case that a technological development of an exhaust gas decrease is not in time. For example, regarding the NMOG out of the exhaust gas regulation in California State, it is intended that a manufacturer and vehicle, who use a DOR (Direct Ozone Reduction) for the vehicle, can receive an NMOG approval. In other words, because ozone (O₃), a main component of photochemical smog, occurs due to a chemically change of the NOx and the NMOG by the sun light, California State makes it a rule to give a predetermined benefit (NMOG credit), which assumes that an exhaust amount of the NMOG is achieved, to a vehicle that runs while decomposing (purifying) the ozone by an ozone decomposition catalyst as the DOR; and to a manufacturer that sells such the vehicle. Therefore, the manufacturer sells a vehicle, for example, with an ozone purifier, whose ozone decomposition catalyst layer is provided on a radiator surface (surface of radiator fins) of the vehicle, as the ozone purifier using the DOR (for example, see pages 3 and 4 and FIG. 1 in Japanese Patent Laid-Open Publication No. 2001-347829).

Whereas, an ozone purification performance of such the ozone purifier is not constant and decreases according to deterioration of a catalyst, a flaking of its layer, and the like. Therefore, the NMOG credit is given, depending on the ozone purification performance after running of 150,000 miles (about 240,000 km). Accordingly, each manufacturer obtains an ozone purification performance value for each kind of vehicle and each model thereof after the running of 150,000 miles, and demands an application of the NMOG credit for the authority at the obtained value or an application value lower than the obtained value. On the other hand, even if a vehicle satisfies the application value when new, there could occur an inadequate situation that the ozone purification performance value exceeds the application value before the running of 150,000 miles. For such the situation, in order to appropriately perform a countermeasure such as maintenance and inspection, the manufacturer must demonstrate the ozone purification performance and durability of the ozone purifier during a total effective usage period of the vehicle, mount an onboard diagnosis apparatus (OBD) for monitoring the performance of the ozone purifier, and assure appropriate exhaust control. Therefore, each manufacturer develops the onboard diagnosis apparatus.

For example, a conventional OBD apparatus has a configuration shown in FIG. 12. it is a simplified section drawing showing the conventional OBD apparatus.

As shown in FIG. 12, the conventional OBD apparatus provides an ozone sensor 201 for measuring an ozone concentration at an upstream side of a radiator, whose surface is coated with an ozone purification catalyst, and an ozone sensor 203 for measuring an ozone concentration at a downstream side of the radiator, measures the ozone concentrations at the upstream and downstream sides through each of an upstream side air sampling tube 203 and a downstream side air sampling tube 204, and sends measurement signals to an ECU (Emission Control Unit).

The ECU calculates an ozone purification performance η, for example, according to an formula (1):
η=1−(C2)/(C1), (1)
based on an upstream side ozone concentration, that is, an ozone concentration C1 in air not passing an ozone purification catalyst layer C, and a downstream side ozone concentration, that is, an ozone concentration C2 in air passing the ozone purification catalyst layer C.

However, because an ozone purifier for a vehicle using the conventional OBD apparatus comes short of accuracy of an ozone sensor in a low concentration and is difficult to handle a concentration property change shown by a change of an off-set value and an ozone concentration sensor correlation line, it becomes difficult to measure an ozone purification ratio with good accuracy. As the result, there is a possibility of resulting in an erroneous detection.

Accordingly, it is strongly requested an ozone purifier for a vehicle whose measurement accuracy of the ozone purification ratio is heightened without improving an ozone sensor by overcoming the defect of the conventional technology.

### SUMMARY OF THE INVENTION

The inventors et al. have devoted themselves to perform a study, considering the problem described above, and as a result, in the ozone purifier for the vehicle mounting the conventional OBD apparatus, have discovered to be able to cite as a factor of lowering ozone sensor accuracy: (1) humidity, (2) adsorption of other gases, (3) displacement of an adsorption balance due to a temperature change, and (4) a change of a concentration property of an ozone sensor such as a resistance change of a semiconductor due to a temperature change and to solve the problem of the present invention by appropriately calibrating the change of the concentration property; and have come to originating the present invention.

A first aspect of the present invention to solve the problem is an ozone purifier for a vehicle comprising: an ozone purification catalyst mounted on the vehicle; a first ozone concentration detection mechanism for detecting an ozone concentration in air before it passing the ozone purification catalyst; a second ozone concentration detection mechanism for detecting an ozone concentration in air after it passing the ozone purification catalyst; a deterioration detection mechanism for using output values of the first and second ozone concentration detection mechanisms and detecting deterioration of the ozone purification catalyst; and furthermore a mechanism for calibrating the output values of the first and second ozone concentration detection mechanisms, wherein when detecting the deterioration of the ozone purification catalyst, the purifier calibrates the output values of the first and second ozone concentration detection mechanisms.

A second aspect of the present invention is an ozone purifier for a vehicle comprising: an ozone purification catalyst mounted on the vehicle; an ozone concentration detection mechanism for detecting an ozone concentration in air before/after its passing the ozone purification catalyst; a deterioration detection mechanism for using an output value of the ozone concentration detection mechanism and detecting deterioration of the ozone purification catalyst; and furthermore a mechanism for calibrating the output value of the ozone concentration detection mechanism, and when detecting the deterioration of the ozone purification catalyst, the purifier calibrates the output value of the ozone concentration detection mechanism. Thus configured, the change of the concentration property in the ozone concentration detection mechanisms can be appropriately calibrated by a simple configuration and measurement accuracy of an ozone purification ratio can be improved without improving the ozone sensor.

Meanwhile, in the present invention each of the first ozone concentration detection mechanism and the second ozone concentration detection mechanism means at least one ozone concentration detection mechanism.

A third aspect related to the present invention is an ozone purifier for a vehicle, wherein a mechanism for calibrating an output value of each of the ozone concentration detection mechanisms comprises a mechanism for contacting air not containing ozone with each of the ozone concentration detection mechanisms, and a processing mechanism for calibrating the output value of each of the ozone concentration detection mechanisms, based on the output value of each of the ozone concentration detection mechanisms for the air not containing the ozone and an output value of each of the ozone concentration detection mechanisms for air sampled from a predetermined intake.

Thus by applying the air not containing the ozone to each of the ozone concentration detection mechanisms as a calibration gas, it can be made to appropriately calibrate a change of a concentration property in the ozone concentration detection mechanisms with a simple configuration.

Meanwhile, when the ozone purifier related to the present invention has the first ozone concentration detection mechanism and the second ozone concentration detection mechanism (the first aspect), “each of the ozone concentration detection mechanisms” described above means the first or second ozone concentration detection mechanism; and when the ozone purifier has an ozone concentration detection mechanism for detecting both ozone concentrations in air before/after it passing an ozone catalyst, “each of the ozone concentration detection mechanisms” described above means the one ozone concentration detection mechanism.

A fourth aspect of the present invention is an ozone purifier for a vehicle, wherein a mechanism for contacting air not containing the ozone with each of the ozone concentration detection mechanisms may comprise a mechanism for storing the air by shielding lights and a mechanism for measuring an elapsed time from a start of the storage.

In addition, a fifth aspect of the present invention is an ozone purifier for a vehicle, wherein a mechanism for contacting air not containing the ozone with each of the ozone concentration detection mechanisms may comprise a mechanism for storing the air, a mechanism for decomposing the ozone in the stored air, and a mechanism for introducing the stored air into each of the ozone concentration detection mechanisms.

A sixth aspect of the present invention is an ozone purifier for a vehicle, wherein a mechanism for contacting air not containing the ozone with each of the ozone concentration detection mechanisms may comprise a mechanism for storing the air not containing the ozone and a mechanism for introducing the stored air into each of the ozone concentration detection mechanisms.

Thus configured, air not containing the ozone can be used as a calibration gas.

### BRIEF DESCRIPTION OF THE DRAWINGS

FIG. 1 is a simplified section drawing showing an ozone purifier for a vehicle related to a first embodiment of the present invention.

FIG. 2A is a drawing showing one example of an ozone purifier main body for a vehicle related to the present invention; and FIG. 2B is an enlarged drawing of a part A in FIG. 2A.

FIG. 3 is a drawing showing one example of an ozone sensor applied to an ozone purifier for a vehicle related to the present invention.

FIGS. 4A, 4B, and 4C are graphs showing concentration property changes of ozone sensors.

FIG. 5 is a drawing showing a change over time of an ozone concentration in air in a storage measure

FIG. 6 is a simplified section drawing showing an ozone purifier for a vehicle related to a variation mode of the first embodiment of the present invention.

FIG. 7 is a simplified section drawing showing an ozone purifier for a vehicle related to a second embodiment of the present invention.

FIG. 8 is a simplified section drawing showing an ozone purifier for a vehicle related to a third embodiment of the present invention.

FIG. 9 is a graph showing a relationship between an ozone concentration and an output value in the second and third embodiments.

FIG. 10 is a simplified section drawing showing an ozone purifier for a vehicle related to a fourth embodiment of the present invention.

FIG. 11 is a drawing showing an ozone measurement manner according to the fourth embodiment.

FIG. 12 is a simplified section drawing showing a conventional OBD apparatus.

### DESCRIPTION OF THE PREFERRED EMBODIMENTS

Here will be described embodiments of the present invention, referring to appended drawings.

### FIRST EMBODIMENT

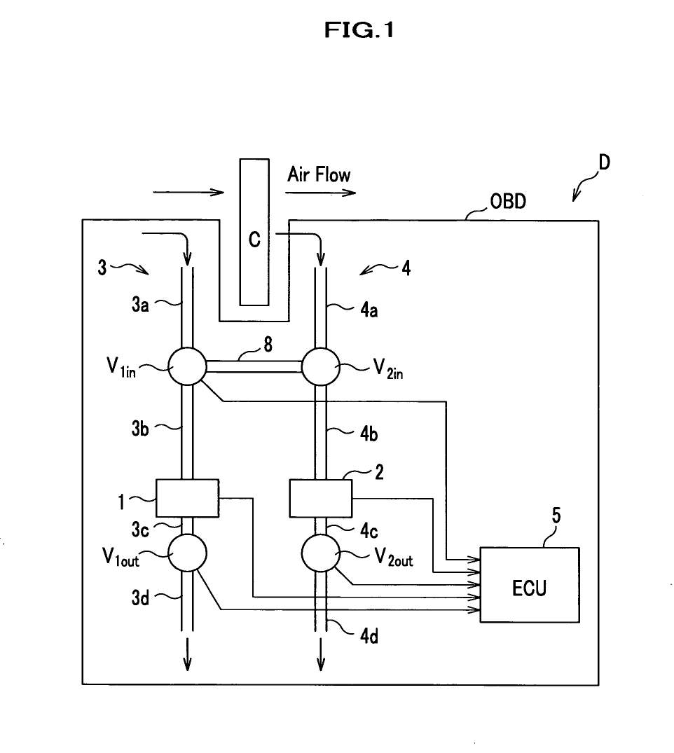

Firstly, an ozone purifier for a vehicle related to a first embodiment of the present invention will be described, based on FIGS. 1 to 5.

Firstly, the ozone purifier for the vehicle related to the first embodiment of the present invention will be generally described, based on FIG. 1.

An ozone purifier D for a vehicle (hereinafter referred to as “for-vehicle ozone purifier D” as needed) is as shown in FIG. 1 mainly configured of a ozone purifier main body C for decomposing ozone in air by running the vehicle and passing the air, and an onboard diagnosis apparatus OBD for monitoring an ozone purification performance of the ozone purifier main body C.

In a preferred embodiment the ozone purifier main body C may be configured, for example, by providing an ozone catalyst layer on a surface (surface of radiator fins) of a vehicle radiator.

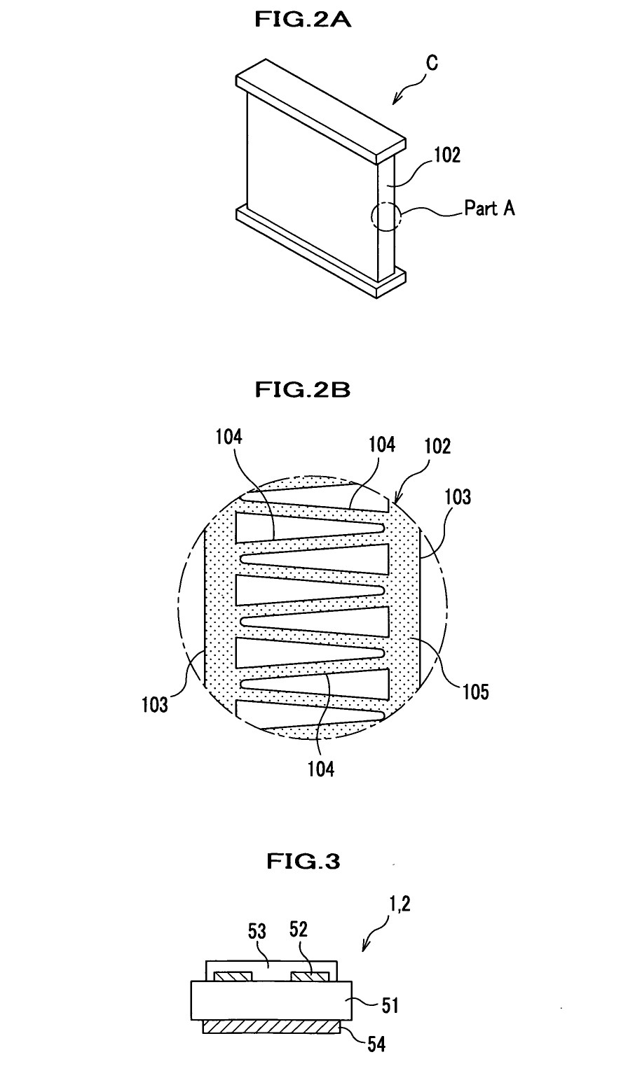

In other words, as shown in FIGS. 2A and 2B, at a front side of a passenger car of a vehicle is placed a radiator 102 as a heat exchanger, and the radiator 102 is mainly configured of a plurality of cooling pipes 103, where cooling liquid downwardly flows from up to down, and a plurality of radiator fins 104 fixedly provided between each of the cooling pipes 103. And the radiator 102 discharges heat of the cooling liquid of an engine heated during running of the vehicle and the like into air and thereby always keeps the cooling liquid at an appropriate temperature.

Here on both outer surfaces of the cooling pipes 103 and the radiator fins 104 is coated an ozone purification catalyst layer 105 over the whole surfaces, using an adhesion measure such an organic binder (not shown). And the catalyst layer 105 decomposes (purifies) ozone (O₃) into oxygen (O₂) in the running, which ozone is a chemical substance in air passing between each of the radiator fins 104 of the radiator 102, and thus decreases the ozone in the air (see FIGS. 2A and 2B).

However, in the present invention such the ozone purifier main body C is not specifically limited if it can decompose and dispose the ozone in the air into the oxygen by the running of the vehicle.

In other words, as shown in FIG. 1, if the ozone in the air is decomposable and disposable into the oxygen by the air passing from an upstream side of the ozone purifier main body C, the purifier main body C is not specifically limited.

The onboard diagnosis apparatus OBD is a detection apparatus for monitoring a decomposition performance of a catalyst of such the ozone purifier main body C.

In other words, the catalyst layer 105 in the ozone purifier main body C gradually peels off downward from a higher portion of a temperature, that is, from an upper portion of the radiator 102 downward, accompanying an increase of a running distance and the like. Therefore, the ozone purification performance gradually decreases, accompanying the peel of the catalyst layer 105.

The onboard diagnosis apparatus OBD related to the first embodiment is an apparatus that comprises a first ozone sensor 1 of a first ozone concentration detection mechanism through an upstream side pipe 3 (3a, 3b . . . ) for sampling air at the upstream side of the ozone purifier main body C, and a second ozone sensor 2 of a second ozone concentration detection mechanism through a downstream side pipe 4 (4a, 4b . . . ) for sampling air at a downstream side of the ozone purifier main body C; and that sends an ECU 5 as electric signals an output value (X1) of the first ozone sensor 1 corresponding to an ozone concentration in the air at the upstream side, where ozone detected from the first ozone sensor 1 is not decomposed, and an output value (X2) of the second ozone sensor 2 corresponding to an ozone concentration in the air at the downstream side, where ozone detected from the second ozone sensor 2 is decomposed by the ozone purifier main body C, wherein the ECU 5 measures an ozone purification performance in real time or intermittently by calibrating these output.

In other words, the ECU 5 calculates an ozone purification performance η, for example, according to a formula (1):
η=1−(X′2)/(X′1). (1)
In the formula the X′1 and the X′2 are respective values after the calibration of the output values of the first ozone sensor 1 and the second ozone sensor 2.

For example, as shown in FIG. 3, in any of the first ozone sensor 1 and the second ozone sensor 2 are arranged a sensor layer 53 on one face (upper face in FIG. 3) of a substrate 51 through a conducting wire 52, and a heater 54 on the other face thereof for a purpose of improving reactivity. The sensor layer 53 is, for example, formed of a semiconductor particle of such a sintered body of an indium tin oxide (ITO) particle. In the semiconductor particle, on a surface of a secondary particle of a several nano-microns order are deposited crystal particles of a several nano-meters order. If an oxide gas (ozone) adsorbs in such a crystal, it is thought that a trap layer of electrons occurs and thereby electric resistance increases.

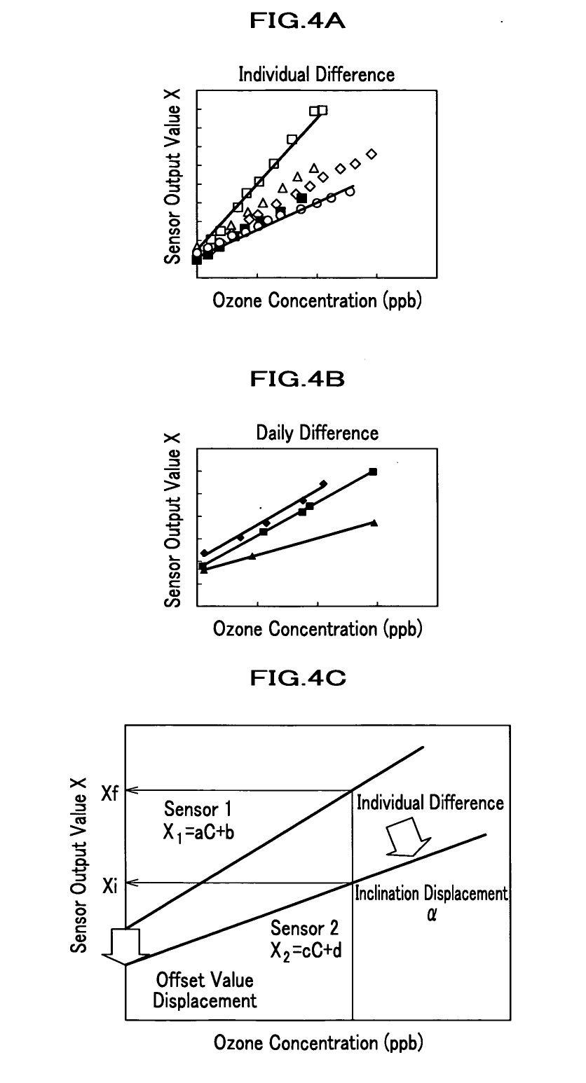

The first ozone sensor 1 and the second ozone sensor 2 having such the composition are, as shown in FIG. 4A, different in sensitivity according to an individual difference of the ozone sensors, and a change of a concentration property occurs according to a vehicle driving condition, an atmosphere in stopping a vehicle such as parking, and the like.

[Individual Difference]

In other words, for example, an ozone sensor can compensate a concentration inclination by actually measuring a gas containing a known predetermined concentration of ozone, thus absorb an individual difference, and accurately measure an ozone concentration.

To be more precise, it can be made to calibrate an inclination of a sensitivity ratio by multiplying an inclination between an ozone concentration (ppb) of a standard and an output value (X) of an ozone sensor by a coefficient A. Meanwhile, it is preferable to calibrate the sensitivity ratio due to the individual difference of such the first ozone sensor 1 and the second ozone sensor 2 before building in the for-vehicle ozone purifier D related to the present invention, particularly the onboard diagnosis apparatus OBD.

Similarly as shown in FIG. 4A, although in an offset value the individual difference also occurs for each ozone sensor, it is also preferable to perform calibration before building in the for-vehicle ozone purifier D related to the present invention, specifically the onboard diagnosis apparatus OBD.

[Concentration Property of Ozone Sensor]

In addition, as shown in FIG. 4B, because in the first ozone sensor 1 and the second ozone sensor 2 the concentration property changes not only by such the individual difference but also by various environmental factors, it becomes necessary to calibrate the concentration property. In other words, the concentration property of the first ozone sensor 1 and the second ozone sensor 2 changes due to various factors such as humidity (adsorption of water), adsorption of other gases such as an oxidizing gas and a reductive gas, deterioration over years, heat, an exposure to toxic substances, being left without turning on electricity, and adhesion of such dust.

Therefore, there occurs a need for calibrating the ozone sensor corresponding to the change of the concentration property due to these factors.

In the present invention, in order to perform such the calibration, it is necessary to calibrate the concentration property of the first ozone sensor 1 and the second ozone sensor 2 by contacting a gas not containing ozone (hereinafter referred to as zero gas) with them.

In the present invention the calibration of such the concentration property of the first ozone sensor 1 and the second ozone sensor 2 is performed by contacting the gas not containing the ozone with them by a predetermined amount.

Although if such the zero gas not containing ozone is a gas insensitive (inactive) for an ozone sensor, it is not specifically limited, the zero gas is preferably the gas based on air such one sealed in a container for a predetermined time or preferably in the container with a light blocking effect and another air where the ozone is decomposed by an ozone decomposition catalyst from a viewpoint of the zero gas being easily obtained.

For example, as shown in FIG. 5, when sealing air in a storage measure such as a tank, an ozone concentration in the air decreases as time passes, and becomes zero, for example, after three hours. That is, such the air storage measure can be a zero gas production measure. At this time a care should be taken for an air composition sealed in the air storage measure, a wall material thereof, a ratio of a gas volume and a wall area, a temperature, and the like. In other words, a zero gas production time (time necessary for the ozone concentration becoming zero) should be investigated in advance for each of such a specific air storage measure. In addition, because the ozone occurs in the atmosphere by a photochemical reaction, it is preferable to select a material with the light blocking effect as that of the air storage measure.

Again described based on FIG. 1, the onboard diagnosis apparatus OBD in the for-vehicle ozone purifier D has a following configuration:

The pipe 3 for passing air before ozone purification through the first ozone sensor 1 comprises the upstream side pipe 3a of the first ozone sensor 1 separated by a three directional valve V1in, and the pipe 3b between the three directional valve V1in and the first ozone sensor 1, a pipe 3c at an outlet side of air from the first ozone sensor 1, and a pipe 3d at an outlet side of a valve V1out, which pipes 3c and 3d are separated by the valve V1out for blocking an exhaust flow channel of the first ozone sensor 1. Meanwhile, the three directional valve V1in is a three directional valve having an open/close function.

Similarly the pipe 4 comprises the upstream side pipe 4a of the second ozone sensor 2 separated by a three directional valve V2in, and the pipe 4b between the three directional valve V2in and the second ozone sensor 2, a pipe 4c at an outlet side of air from the second ozone sensor 2, and a pipe 4d at an outlet side of a valve V2out, which pipes 4c and 4d are separated by the valve V2out for blocking an exhaust channel of the second ozone sensor 2. Meanwhile, the three directional valve V2in is a three directional valve that can change a flow channel for passing through the pipes 4a and 4b and that for passing through a pipe 8 and the pipe 4b.

And between the three directional valves V1 in and V2in is provide the pipe 8 of a bypass flow channel.

To the ECU 5 are respectively electrically connected the first ozone sensor 1, the second ozone sensor 2, and the valves V1in, V1out, V2in, and V2out. The ECU 5 comprises a function of memorizing time from an engine stop to the next engine start; a function of controlling an open/close and change of the valves V1in, V1out, V2in, and V2out; a function of compensating output values of the first ozone sensor 1 and the second ozone sensor 2; and a function of detecting deterioration of the ozone purifier main body C, based on the compensation output values.

[Operation]

Here will be described an operation of the onboard diagnosis apparatus OBD of the first embodiment thus configured.

[At Time of Engine Stop]

In order to make space between the valves V1in and V1out and space between the valves V2in and V2out zero gas space containing no ozone at time of stopping an engine, the ECU 5 firstly closes the valve V1in, thereby changes the valve V2in to a side of the pipe 8, and thereby close the valves V1out and V2out. Thus changing the valves, air can be confined in the space between the valves V1in and V1out and the space between the valves V2in and V2out. The ECU 5 memorizes time at the time of the engine stop.

[At Time of Engine Start]

Next at time of starting the engine the ECU 5 determines from the time at the last engine stop whether or not air introduced into the space between the valves V1in and V1out and the space between the valves V2in and V2out has become the zero gas.

If the ECU 5 determines that the air introduced into each the space has not become the zero gas, it does not perform the open/close and change operations of the valves because there is a possibility of erroneous detection. After the engine start if the ECU 5 determines that the air introduced into each the space has become the zero gas, it calibrates the first ozone sensor 1 and the second ozone sensor 2 as follows:

[Compensation of Output Value]

Firstly, the ECU 5 calibrates offset values of the first ozone sensor 1 and the second ozone sensor 2. In other words, as shown in FIG. 4C, because output values of the first ozone sensor 1 and the second ozone sensor 2 linearly increase for an ozone concentration, the ECU 5 first makes it zero respective intercepts of lines of the first ozone sensor 1 and the second ozone sensor 2 for the vertical axis, and next compensates respective inclinations thereof. For example, assuming that the inclination of the line of the first ozone sensor 1 is “a” and the intercept for the vertical axis is “b” when the ozone concentration is zero, the inclination of the line of the second ozone sensor 2 is “c” and the intercept the vertical axis is “d” when the ozone concentration is zero, and the ozone concentration is “C,” a relationship between the ozone concentration and the output value (X1) in the first ozone sensor 1 is a linear function expressed in a formula: X1=aC+b, and a relationship between the ozone concentration and the output value (X2) in the second ozone sensor 2 is a linear function expressed in a formula: X2=cC+d.

Therefore, by obtaining a ratio of the offset values (b, d) and that of the inclinations of the first ozone sensor 1 and the second ozone sensor 2, the output values thereof can be calibrated.

To be more precise, the ECU 5 subtracts “b” from an output value of the first ozone sensor 1 and “d” from that of the second ozone sensor 2, and multiplies the respective obtained values by an inclination ratio “A” of the first ozone sensor 1 and the second ozone sensor 2.

In the first embodiment, in order to obtain the inclination ratio “A” of the first ozone sensor 1 and the second ozone sensor 2, the ECU 5 exposes them under an environment of a same ozone concentration.

Therefore, the ECU 5 opens the valves V1in, V1out, and V2out, and thereby makes the valve 2in at the side of the pipe 8. Thus setting the valves, non purified air, that is, air containing a same concentration of ozone, results in passing through the first ozone sensor 1 and the second ozone sensor 2.

[Determination of Ozone Purifier]

Thus after an end of the calibration of the first ozone sensor 1 and the second ozone sensor 2 (at time of running the engine), the ECU 5 changes the valve V2in to the side of the pipe 4a, blocks air from flowing to the side of the second ozone sensor 2 before the air passing the ozone purifier main body C, and thereby passes air through the second ozone sensor 2 after the air passing the ozone purifier main body C.

Thus controlling each of the valves, the first ozone sensor 1 measures an ozone concentration in the air before its passing the ozone purifier main body C and sends the measurement result to the ECU 5.

On the other hand, the second ozone sensor 2 measures an ozone concentration in the air after its passing the ozone purifier main body C and sends the measurement result to the ECU 5. The ECU 5 determines whether or not the ozone purifier main body C has deteriorated, based on the output values from the first ozone sensor 1 and the second ozone sensor 2, which values correspond to the ozone concentrations in the air before/after its passing through the ozone purifier main body C.

In other words, the ozone purification performance η can be calculated, for example, according to the formula (1):
η=1−(C2)/(C1), (1)
where C1 is an ozone concentration in air before ozone purification and C2 is an ozone concentration in air after the ozone purification. Here assuming that a ratio of the ozone concentration (C1) in the air before the ozone purification and the ozone concentration (C2) in the air after the ozone purification is a ratio of values after calibration of an output value of the first ozone sensor 1 and that of the second ozone sensor 2, the ozone purification performance η, that is, an ozone deterioration ratio, can be calculated.

Meanwhile, actually after the first ozone sensor 1 and the second ozone sensor 2 becoming stable, the ECU 5 makes it X1 an output value of the first ozone sensor 1, X2 an output value of the second ozone sensor 2, “b” the offset value of the first ozone sensor 1, and “d” the offset value of the second ozone sensor 2; thereby makes the ozone purification performance η:
η=1−(a−(X2−d)/(c(X1−B); (1′)
and thus monitors the ozone purification performance η.

Here when the performance η becomes lower than a predetermined value, the ECU 5 can send a signal so that a MIL (malfunction indicator light) is lit, and inform a driver of an ozone purification catalyst in the ozone purifier main body C having deteriorated.

Meanwhile, when performing nothing but normal determination of the ozone purification catalyst in the ozone purifier main body C, it is not necessary to wait till the output value of the second ozone sensor 2 becomes stable. In other words, when monitored, the previous formula (1) continues on indicating 0 before the valve V2in is changed. Here when the valve V2in is changed, the ozone purification ratio expressed in the formula (1) continues on ascending and tries to converge on a predetermined value. Accordingly, when a value of the formula (1) exceeds the predetermined value just after the change of the valve V2in, the ozone purifier main body C can be determined to be normal. Thus configured, the determination can be speedily performed.

On the other hand, when the value of the formula (1) does not exceed the predetermined value just after the change of the valve V2in, the ECU 5 again performs determination by waiting for the second ozone sensor 2 becoming stable.

[Variation Mode]

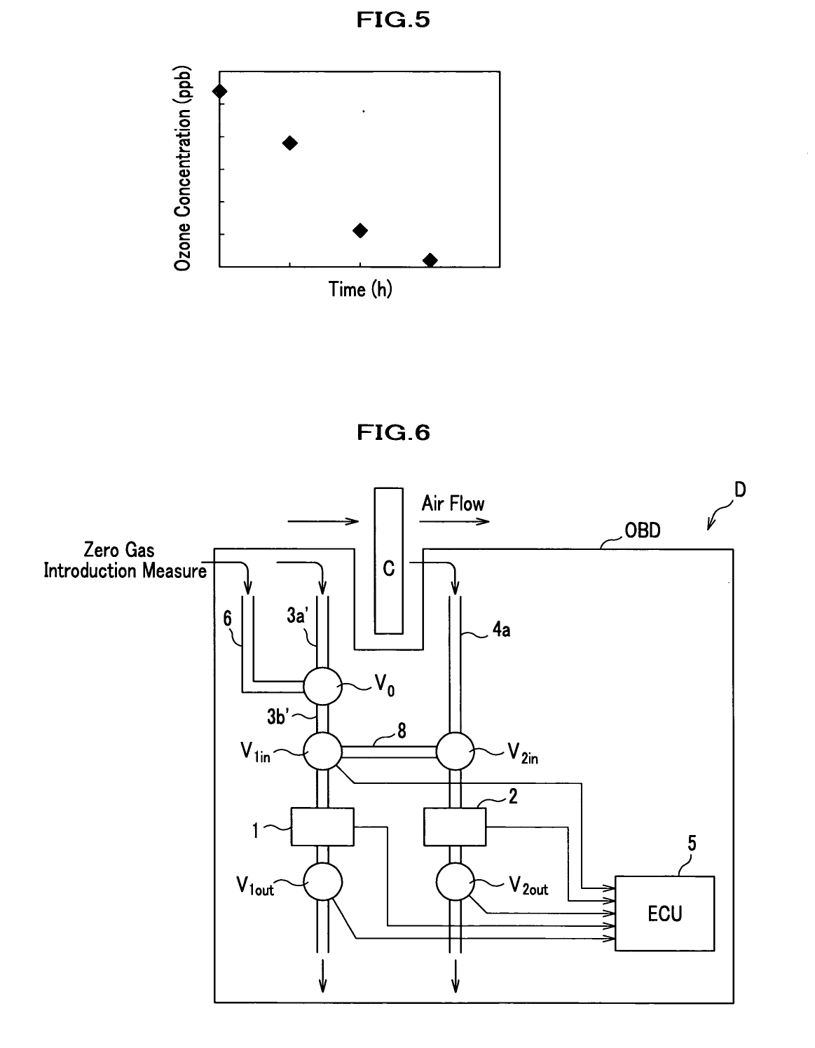

Meanwhile, the first embodiment shown in FIG. 1 can also be changed, for example, as shown in FIG. 6. A mode shown in FIG. 6 further comprises, in the first embodiment shown in FIG. 1, a pipe 6 for introducing the zero gas from a zero gas introduction measure (not shown) at a more upstream side of the valve V1in, and a valve V0 for changing a pipe 3a′ and the pipe 6.

The embodiment of FIG. 6 is different from that of FIG. 1 in a point of using the zero gas from the zero gas introduction measure instead of using air from the side of the ozone purifier main body C at time of a vehicle stop. Thus configured, it can be made to speedily calibrate the first ozone sensor 1 and the second ozone sensor 2 without waiting till ozone in air decomposes.

### SECOND EMBODIMENT

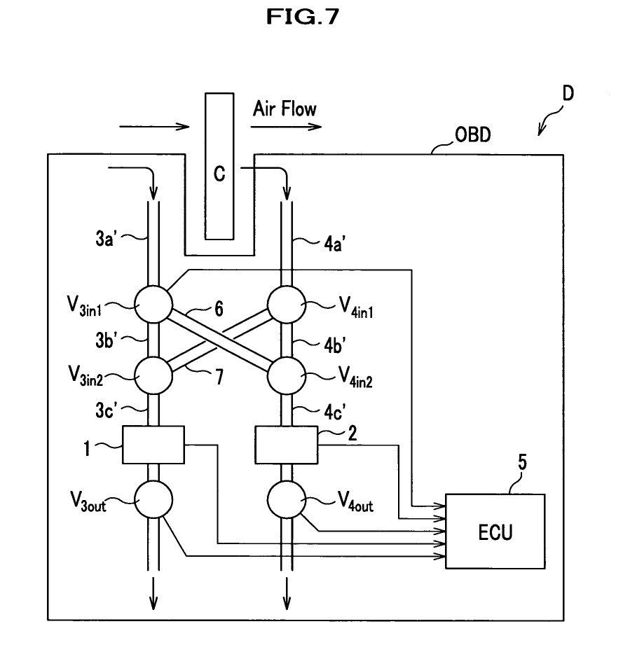

Here will be described a second embodiment, referring to FIGS. 7 and 9.

The embodiment is different from that of FIG. 1 in an arrangement of valves and piping, and a measurement method of the purification ratio. Accordingly, to same elements as in the first embodiment shown in FIG. 1 are appended same symbols, and descriptions thereof are omitted. Meanwhile, in FIG. 7 a valve V4in1 is a three-directional valve whose three directions are always opened; a valve V4in2 is a three-directional valve that can change a flow channel for communicating a pipe 4b′ with a pipe 4c′ and a flow channel for communicating the pipe 6 with the pipe 4c′; and a valve V3in2 is a three-directional valve that can change a flow channel for communicating the pipe 3b′ with a pipe 3c′ and a flow channel for communicating a pipe 7 with the pipe 3c′.

As shown in FIG. 7, the for-vehicle ozone purifier D related to the second embodiment differs in a point of comprising two systems of air introduction flow channels for calibrating output values of the first ozone sensor 1 and the second ozone sensor 2.

Because the ECU 5 has a mechanism for memorizing time from an engine stop till the next engine start, it can determine whether or not a sealed-in gas has become the zero gas, based on the memorized time. Together with the engine stop, the ECU 5 makes a state of changing the valve V4in2 to the side of the pipe 6 and the valve V3in2 to the side of the pipe 3′b; closes the valves 3in1, V3out, and V4out; and thus confines a gas. At time of the next engine start, the ECU 5 determines whether an elapsed time from the last engine stop is longer or shorter than a zero gas production time. When longer, the ECU 5 makes a sensor output value an offset value. When shorter, the ECU 5 does not perform a measurement because there is a possibility of an erroneous detection. Supposing that the elapsed time reaches the zero gas production time during running, if it is after completion of the sensor activation, the ECU 5 makes it the offset value an output value in reaching the zero gas production time; and if it is not after the completion of the sensor activation, the ECU 5 makes an output value then the offset value.

When using a method of introducing the zero gas into the first ozone sensor 1 and the second ozone sensor 2 same as in the first embodiment, the output value after the completion of the sensor activation can be made the offset value without depending on the elapsed time from the last stop time, using a zero gas bottle and a zero gas production apparatus.

The ECU 5 opens the open/close the valves V3in, V3out, and V4out; changes the V4in2 to the side of the pipe 4′b; and introduces upstream side air into the first ozone sensor 1 and downstream side air into the second ozone sensor 2. After the change of the flow channels, the ECU 5 waits till the output values of the first ozone sensor 1 and the second ozone sensor 2 become stable. Assume that respective the output values of the first ozone sensor 1 and the second ozone sensor 2 are X1 and X2. Then assuming that the offset values of the first ozone sensor land the second ozone sensor 2 set as described before are respectively “b” and “d,” an apparent purification ratio ηa is expressed in a formula ( at an interval A):
ηa=1−(α(X2−d))/(γ(X1−b)).

The ECU 5 changes the valve V3in2 to the side of the pipe 7 and the valve V4in2 to the side of the pipe 6; and introduces the upstream side air into the second ozone sensor 2 and the downstream side air into the first ozone sensor 1. After the change of the flow channels, the ECU 5 waits till the output values of the first ozone sensor 1 and the second ozone sensor 2 become stable. Assuming that respective output values of the first ozone sensor 1 and the second ozone sensor 2 are X1′ and X2′, an apparent purification ratio ηb is expressed in a formula (at an interval B):
ηb=1−(γ(X1′−b))/(α(X2′−d)).

A relationship between the output values X1, X2, X1′, and X2′ and an ozone concentration is as in FIG. 9; and the ηa and the ηb do not indicate a true ozone purification ratio and become different values, respectively. Assuming that the true ozone purification ratio at the intervals A and B is equal and is ηt, because the ηa, ηb, and ηt satisfy a formula (2) below, the true ozone purification ratio ηt can be measured by obtaining the ηa and the ηb:
ηt=1−((1−ηa)(1−ηb))^{1/2}. (2)

A condition that the true ozone purification ratio is equal at such the intervals A and B is, for example, one that a water temperature of a radiator and a vehicle speed are both constant.

### THIRD EMBODIMENT

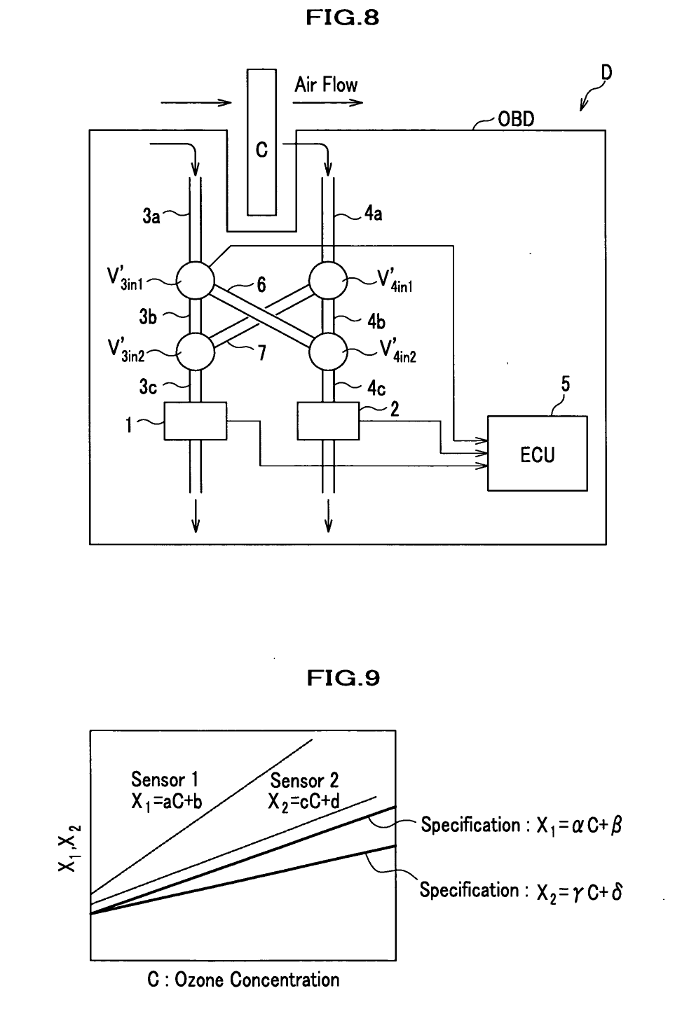

Here will be described a third embodiment, based on FIGS. 8 and 9.

This embodiment is different from the second embodiment shown in FIG. 7 in points that: the valves V′3out and V′4out at a downstream side of the first ozone sensor 1 and the second ozone sensor 2 are not respectively provided; the valve V′3in1 is a three-directional valve opened to flow channels in three directions; and a method of measuring the purification ratio is different. Accordingly, to same elements as in the second embodiment shown in FIG. 7 are appended same symbols, and descriptions thereof are omitted.

The ECU 5 changes a valve V′3in2 to the side of the pipe 3b′ and a valve V′4in2 to the side of the pipe 4b′; and introduces upstream side air into the first ozone sensor 1 and downstream side air into the second ozone sensor 2. Assume that an interval till flow channels are changed is the interval A. After the change of the flow channels, the ECU 5 waits till output values of the first ozone sensor 1 and the second ozone sensor 2 become stable. Assume that respective output values of the first ozone sensor 1 and the second ozone sensor 2 after becoming stable are X1A and X2A. At this time, assume that concentration properties of the first ozone sensor 1 and the second ozone sensor 2 deviate from specification properties, respectively, as in FIG. 9. At this time a true concentration is expressed in a formula of (X1A−b)/a at the upstream side and a formula of (X2A−d)/c at the downstream side (at the interval A).

Then the ECU 5 changes the valve V′3in2 to the side of the pipe 7 and the valve V′4in2 to the side of the pipe 6; and introduces the upstream side air into the second ozone sensor 2 and the downstream side air into the first ozone sensor 1. Assume that an interval till flow channels are changed is the interval B. After the change of the flow channels, the ECU 5 waits till the output values of the first ozone sensor 1 and the second ozone sensor 2 become stable. Assuming that respective output values of the first ozone sensor 1 and the second ozone sensor 2 after becoming stable are X1B and X2B, a true concentration is expressed in a formula of (X2B−d)/c at the upstream side and a formula of (X1B−b)/a at the downstream side (at the interval B).

When the ozone purification ratio of the interval A and that of the interval B are equal, a formula (3) below consists:
(a(X2A−d))/(c(X1A−b))=(c(X1B−b))/(a(X2B−d)). (3)

Next, the ECU 5 changes the valve V′3in2 to the side of the pipe 3b′ and the valve V′4in2 to the side of the pipe 4b′; and introduces the upstream side air into the first ozone sensor 1 and the downstream side air into the second ozone sensor 2. Assume that an interval till flow channels are changed is an interval C, and the ECU 5 similarly obtains output values X1C and X2C.

In addition, the ECU 5 changes the valve V′3in2 to the side of the pipe 7 and the valve V′4in2 to the side of the pipe 6; and introduces the upstream side air into the second ozone sensor 2 and the downstream side air into the first ozone sensor 1. Assume that an interval till flow channels are changed is an interval D, and the ECU 5 similarly obtains output values X1D and X2D.

Assuming that respective output values of the first ozone sensor 1 and the second ozone sensor 2 after becoming stable are X1B and X2B, the true concentration is expressed in a formula of (X2B−d)/c at the upstream side and a formula of (X1B−b)/a at the downstream side.

When the ozone purification ratio of the interval C and that of the interval D are equal, a formula (4) below consists:
(a(X2C−d))/(c(X1C−b))=(c(X1D−b))/(a(X2D−d)). (4)

By obtaining numerals of a, c, b, and d from the formulas (3) and (4), the true purification ratio can be obtained.

### FOURTH EMBODIMENT

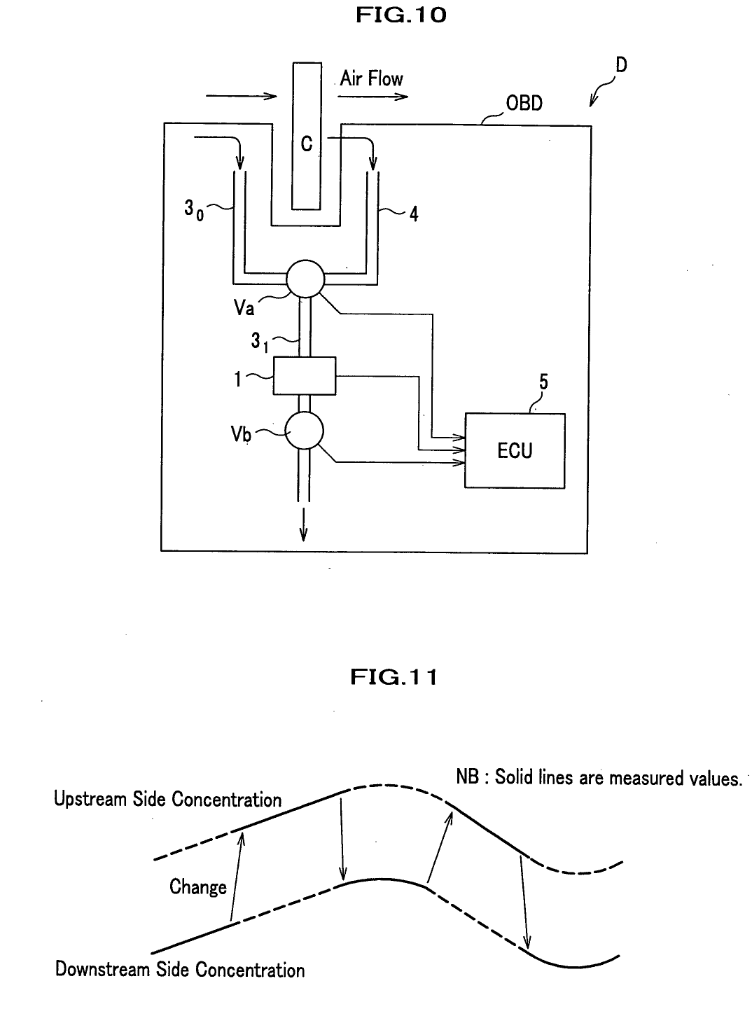

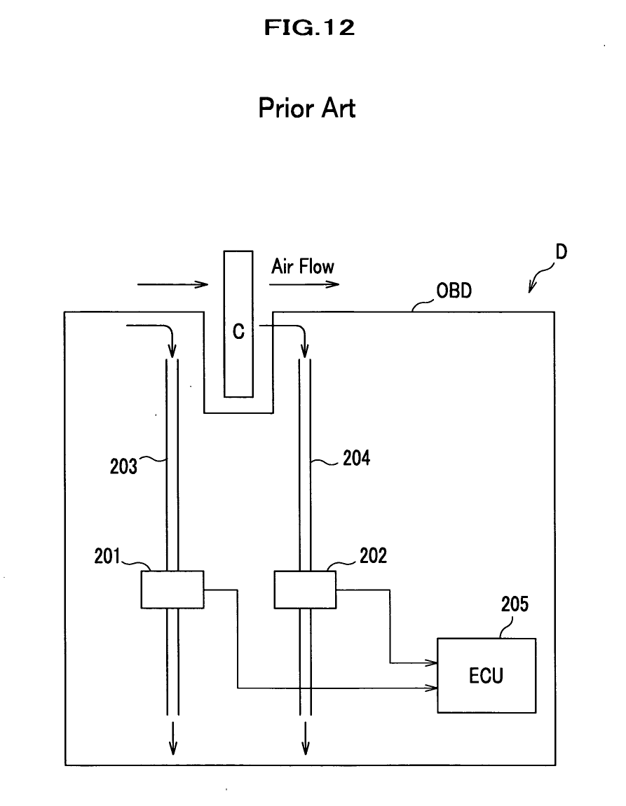

Next will be described a fourth embodiment, based on FIGS. 10 and 11.

As shown in FIG. 10, the embodiment measures an ozone concentration in air before/after its passing the purifier main body C, using one ozone sensor 1.

Therefore, the for-vehicle ozone purifier D has a configuration of periodically changing a pipe 3o at the upstream side and the pipe 4 at the downstream side by a valve Va and performing a measurement by the ozone sensor 1.

Because the ECU 5 has a memory mechanism for memorizing time from an engine stop to the next engine start, the ECU 5 determines whether or not a gas sealed in same as in the first and second embodiments has become the zero gas, based on the memorized time. Together with the engine stop, the ECU 5 closes the valves Va and Vb, and confines a gas. At time of the next engine start, the ECU 5 determines whether an elapsed time from the last engine stop is longer or shorter than a zero gas production time. When longer, the ECU 5 makes it an offset value an output value of the ozone sensor 1 after an activation completion thereof. When shorter, the ECU 5 does not perform a measurement because there is a possibility of an erroneous detection. Supposing that the elapsed time reaches the zero gas production time during driving, if it is after the activation completion of the ozone sensor 1, the ECU 5 makes it the offset value an output value in reaching the zero gas production time; and if it is not after the activation completion of the ozone sensor 1, the ECU 5 makes an output value then the offset value.

In addition, when using a method of introducing the zero gas into the first ozone sensor 1 same as in the first and second embodiments, the output value after the activation completion of the ozone sensor 1 can be made the offset value without depending on the elapsed time from the last stop time, similarly using a zero gas bottle and a zero gas production apparatus.

The ECU 5 opens the valves Va and Vb, changes the valve Va so as to periodically introduce upstream side air and downstream side air, and monitors the output value of the ozone sensor 1. An image of the output value of the ozone sensor 1 then is as shown in FIG. 11.

An output value corresponding to an ozone concentration in air at a non detection side is estimated by connecting output values measured in a vicinity (dotted line portions in FIG. 11 are approximated by lines connecting solid line ends at front and back of the dotted lines). Assuming that at a time t respective estimation values corresponding to a measurement value X1t at the upstream side and a measurement value X2t at the downstream side are X′1t and X′2t, the ozone purification ratio can be expressed in a formula of (1−(X2t−b))/(X′1t−b) when the ozone sensor 1 monitors the downstream side; and in a formula of (1−(X′2t−b))/(X1t−b) when the ozone sensor 1 monitors the upstream side.

Thus, although the embodiments of the present invention are described, the invention is not limited thereto and is widely applied.

For example, the OBD itself and the calibration method of the detection mechanisms in a vehicle are also within a range of the present invention.

1. An ozone purifier for a vehicle comprising:
an ozone purification catalyst mounted on the vehicle;
a first ozone concentration detection mechanism for detecting an ozone concentration in air before the air passing said ozone purification catalyst;
a second ozone concentration detection mechanism for detecting an ozone concentration in air after the air passing said ozone purification catalyst;
a deterioration detection mechanism for using output values of said first and second ozone concentration detection mechanisms and detecting deterioration of said ozone purification catalyst; and furthermore
a mechanism for calibrating the output values of said first and second ozone concentration detection mechanisms,
wherein when detecting the deterioration of said ozone purification catalyst, said purifier calibrates the output values of said first and second ozone concentration detection mechanisms.

2. An ozone purifier for a vehicle comprising:
an ozone purification catalyst mounted on the vehicle;
an ozone concentration detection mechanism for detecting an ozone concentration in air before/after the air passing said ozone purification catalyst;
a deterioration detection mechanism for using an output value of said ozone concentration detection mechanism and detecting deterioration of said ozone purification catalyst; and furthermore
a mechanism for calibrating the output value of said ozone concentration detection mechanism,
wherein when detecting the deterioration of said ozone purification catalyst, said purifier calibrates the output value of said ozone concentration detection mechanism.

3. An ozone purifier for a vehicle according to claim 1, wherein a mechanism for calibrating an output value of each of said ozone concentration detection mechanisms comprises a mechanism for contacting air not containing ozone with each of said ozone concentration detection mechanisms, and a processing mechanism for calibrating the output value of each of said ozone concentration detection mechanisms, based on the output value of each of said ozone concentration detection mechanisms for the air not containing said ozone and an output value of each of said ozone concentration detection mechanisms for air sampled from a predetermined intake.

4. An ozone purifier for a vehicle according to claim 2, wherein a mechanism for calibrating an output value of said ozone concentration detection mechanism comprises a mechanism for contacting air not containing ozone with said ozone concentration detection mechanism, and a processing mechanism for calibrating the output value of said ozone concentration detection mechanism, based on the output value of said ozone concentration detection mechanism for the air not containing said ozone and an output value of said ozone concentration detection mechanism for air sampled from a predetermined intake.

5. An ozone purifier for a vehicle according to claim 3, wherein a mechanism for contacting air not containing said ozone with each of said ozone concentration detection mechanisms comprises a mechanism for storing the air by shielding lights and a mechanism for measuring an elapsed time from a start of the storage.

6. An ozone purifier for a vehicle according to claim 4, wherein a mechanism for contacting air not containing said ozone with said ozone concentration detection mechanism comprises a mechanism for storing the air by shielding lights and a mechanism for measuring an elapsed time from a start of the storage.

7. An ozone purifier for a vehicle according to claim 3, wherein a mechanism for contacting air not containing said ozone with each of said ozone concentration detection mechanisms comprises a mechanism for storing the air, a mechanism for decomposing the ozone in the stored air, and a mechanism for introducing the stored air into each of said ozone concentration detection mechanisms.

8. An ozone purifier for a vehicle according to claim 4, wherein a mechanism for contacting air not containing said ozone with said ozone concentration detection mechanism comprises a mechanism for storing the air, a mechanism for decomposing the ozone in the stored air, and a mechanism for introducing the stored air into said ozone concentration detection mechanism.

9. An ozone purifier for a vehicle according to claim 3, wherein a mechanism for contacting air not containing said ozone with each of said ozone concentration detection mechanisms comprises a mechanism for storing the air not containing the ozone and a mechanism for introducing the stored air into each of said ozone concentration detection mechanisms.

10. An ozone purifier for a vehicle according to claim 4, wherein a mechanism for contacting air not containing said ozone with said ozone concentration detection mechanism comprises a mechanism for storing the air not containing the ozone and a mechanism for introducing the stored air into said ozone concentration detection mechanism.
